(19) United States
(12) Patent Application Publication
(10) Pub. No.: US 2024/0383946 A1
(43) Pub. Date: Nov. 21, 2024

(54) ARGININE WASH IN PROTEIN PURIFICATION

(71) Applicant: H. LUNDBECK A/S, Valby (DK)

(72) Inventors: Kathleen Schneider, Bremerton, WA (US); Ethan Ojala, Snohomish, WA (US); Sam Marzolf, Bothell, WA (US)

(21) Appl. No.: 18/534,843

(22) Filed: Dec. 11, 2023

Related U.S. Application Data
(60) Provisional application No. 63/591,036, filed on Oct. 17, 2023.
(60) Provisional application No. 63/387,008, filed on Dec. 12, 2022.

(51) Int. Cl. C07K 1/22; C07K 16/18

(52) U.S. Cl. CPC C07K 1/22; C07K 16/18; C07K 2317/24

(57) ABSTRACT

The present invention relates to methods for isolating a product and/or reducing impurities such as Host Cell Proteins (HCP) from a load fluid comprising the product and one or more impurities by passing the load fluid through a medium, followed by at least one wash solution comprising arginine or an arginine derivative at a concentration above about 525 mM and a pH above 8, and collecting the product using an elution solution.

### RELATED APPLICATIONS

The present application claims benefit of priority to U.S. Provisional Application Nos. 63/387,008 filed Dec. 12, 2022 and 63/591,036 filed Oct. 17, 2023, the contents both of which are incorporated by reference in their entireties, the contents both of which are incorporated by reference in their entireties.

### REFERENCE TO AN ELECTRONIC SEQUENCE LISTING

The contents of the electronic sequence listing (11432570012002.xml; Size: 31,505 bytes; and Date of Creation: Dec. 9, 2023) is herein incorporated by reference in its entirety.

### FIELD OF THE INVENTION

The invention relates to methods for isolating a product and/or reducing impurities such as Host Cell Proteins (HCP) from a load fluid comprising the product and one or more impurities by passing the load fluid through a chromatographic column, followed by at least one wash solution comprising arginine or an arginine derivative, e.g., an arginine salt at a concentration above 525 mM and a pH above 8, and collecting the product using an elution solution.

### BACKGROUND OF THE INVENTION

The present invention relates to protein purification and in particular methods for purifying a protein bound to a chromatographic resin by passing at least one wash solution containing arginine or an arginine derivative or an arginine salt through the chromatographic column containing said resin and subsequently collecting the purified protein in the eluate.

By use of recombinant technology, many proteins, such as therapeutic antibodies, are cultured in eukaryotic or prokaryotic host cell lines engineered to express the protein. The use of the desired recombinant protein for pharmaceutical applications is generally contingent on being able to reliably recover adequate levels of the protein from impurities such as host cell proteins, protein variants, and compounds from the culture medium.

Conventional protein purification methods are designed to separate the protein of interest from impurities based on differences in size, charge, solubility, and degree of hydrophobicity. Such methods include chromatographic methods such as affinity chromatography, ion exchange chromatography, size exclusion chromatography, hydrophobic interaction chromatography, immobilized metal affinity chromatography, and hydroxyapatite chromatography. These methods often employ a chromatographic resin that can be designed to selectively adhere either the protein of interest or the impurities. In the bind-elute mode, the desired protein selectively binds to the chromatographic resin and is differentially eluted from the medium by different solvents. In the flow-through mode, the impurities specifically bind to the chromatographic resin while the protein of interest does not, thus allowing the recovery of the desired protein in the “flow-through.”

Current methods for the purification of proteins, such as antibodies, include two or more chromatographic steps. For example, the first step in the protein purification protocol can involve an affinity chromatography step that utilizes a specific interaction between the protein of interest and an immobilized resin. Not only the specific chromatographic columns used have an impact on reducing the impurities, but also the wash and elution conditions. The inventors of the present invention have found that certain concentrations of arginine (or arginine salts or arginine derivatives) and different pH levels used in the washing steps of a chromatographic method results in surprisingly high purity and low amounts of host cell proteins, in particular when applied to the antibodies named herein.

### SUMMARY OF THE INVENTION

The present invention relates to a method for reducing impurities in an eluate comprising a product, the method comprising:
(a) providing a load fluid comprising a product and one or more impurities, wherein the product is an Fc-containing monoclonal antibody,
(b) applying the load fluid in a) to a Protein A chromatography column under conditions suitable for binding the product,
(c) optionally contacting the Protein A chromatography column with a first wash solution,
(d) applying one or more wash solutions (such as 1, 2 or 3 wash solutions) to the Protein A chromatography column, wherein the wash solution comprises arginine or an arginine salt or other arginine derivative in a concentration of above 500 mM, and wherein the pH of the wash solution is greater than 8.0,
(e) optionally contacting the Protein A chromatography column in d) with another second wash solution,
(f) contacting the washed Protein A chromatography column in d) or e) with an elution solution under conditions suitable for eluting the product, and
(g) collecting an eluate comprising the product.

The invention relates to the purification of antibodies, and in particular monoclonal antibodies binding to Calcitonin Gene Related Peptide (CGRP), Pituitary Adenylate Cyclase-Activating Polypeptide (PACAP) and Adrenocorticotropic Hormone (ACTH), such as the antibodies named Eptinezumab, PACAP-1 and ACTH-1 herein below.

### DESCRIPTION OF THE FIGURES

FIG. 1 shows a diagram depicting the results of experiments assaying the HCP (“host cell proteins”) content in a sample following a Protein A column step using variation in arginine concentrations and pH conditions outlined in Example 1.

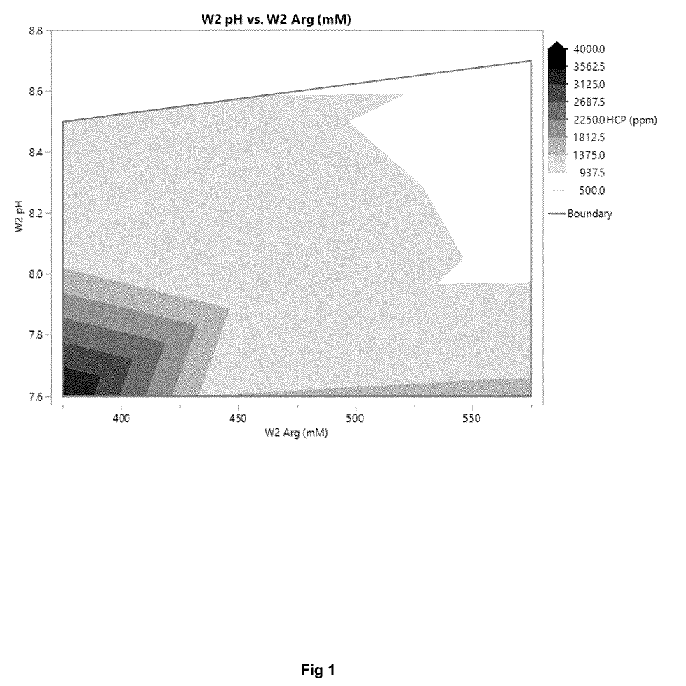

### DESCRIPTION OF THE INVENTION

The present invention provides methods for purifying and recovering a product from a load fluid containing one or more impurities using a purification method that includes an arginine wash or a wash with an arginine salt or arginine derivative. The invention can be applied to the large-scale preparation of proteins for therapeutic and/or diagnostic purposes.

In order for the present invention to be more readily understood, certain terms as used herein are defined. Additional definitions are set forth throughout the detailed description.

The term “arginine derivative” or “arginine salt” herein refers to a derivative resulting from reaction of arginine at the amino group, the carboxy group, or the guanidyl group, or from the replacement of any hydrogen of arginine by a heteroatom, and salts thereof which includes inorganic and/or organic acids such as hydrochloride acid, hydrobromide acid, phosphoric acid, nitrous acid, sulphuric acid, benzoic acid, citric acid, gluconic acid, lactic acid, maleic acid, succinic acid, tartaric acid, acetic acid, propionic acid, oxalic acid, maleic acid, fumaric acid, glutamic acid, pyroglutamic acid, salicylic acid, salicylic acid, saccharin and sulfonic acids, such as methanesulfonic acid, ethanesulfonic acid, toluenesulfonic acid and benzenesulfonic acid. In some embodiements the term “arginine derivative” or “arginine salt” herein refers to and specifically includes Arg·HCl, acetyl arginine, agmatine, arginic acid, N-alpha-butyroyl-L-arginine, or N-alpha-pyvaloyl arginine.

The term “product” refers to a molecule produced by a natural process (e.g. through expression in a mammalian cell such a CHO cell). The term “product” includes a protein, e.g., a therapeutic protein, and in particularly monoclonal antibodies able to bind a protein A resin through its Fc-domain, such as the antibodies named Eptinezumab, PACAP-1 and ACTH-1 herein. The terms “product” and “protein of interest” are used interchangeably.

The term “conditioned culture medium” as used herein refers to the supernatant that is generated from the removal of cells and cellular debris by a separation method, such as centrifugation and/or microfiltration, from cell culture medium that has been exposed to host cells, which may secrete desired products. Conditioned selected nutrients (e.g. vitamins, amino acids, cofactors, and minerals); additional growth factors/supplements including insulin; and additional exogenous, or host cell proteins and impurities. The term conditioned culture medium includes clarified conditioned medium, filtered conditioned medium, and conditioned cell culture medium.

The term “load fluid” refers to a liquid containing the product to be isolated and one or more impurities. A load fluid contacts a chromatographic resin (e.g., is passed through a chromatographic column) under the operating conditions of the invention described below.

The term “impurity” refers to any foreign or undesirable molecule that is present in a solution such as a load fluid. An impurity can be a biological macromolecule such as DNA, RNA, or a protein, other than the protein of interest being purified, that is also present in a sample of the protein of interest being purified. Impurities include, for example, undesirable protein variants, such as aggregated proteins, misfolded proteins, high molecular weight species, low molecular weight species and fragments, and deamidated species; other proteins from host cells that secrete the protein being purified, host cell DNA, components from the cell culture medium, molecules that are part of an absorbent used for affinity chromatography that leach into a sample during prior purification steps, for example, Protein A; an endotoxin; a nucleic acid; a virus, or a fragment of any of the forgoing.

The term “resin” refers to an affinity matrix or resin that can undergo a ligand-biomacromolecule interaction with a product to be isolated during a macromolecular separation process. The resin is preferably a protein A ligand in a Protein A chromatography column.

The term “host cell proteins (HCP)” refers to non-product proteins produced by a host cell during cell culture or fermentation. Accordingly, in some embodiments, an eluate containing a product has HCPs present in less than 100 parts per million (ppm) HCPs (e.g., less than about 50 ppm, or less than about 20 ppm). HCP composition is extremely heterogeneous and dependent on the protein product and purification procedure used. Prior to any marketing approval of a biological product for therapeutic use, the level of contaminating proteins (such as HCPs) in the product must be quantitatively measured according to the ICH and FDA guidelines.

The term “protein” as used herein refers to one or more polypeptides that can function as a unit. The term “polypeptide” as used herein refers to a sequential chain of amino acids linked together via peptide bonds. A therapeutic protein can be, for example, a secreted protein. Therapeutic proteins include antibodies, antigen-binding fragments of antibodies, some of which are described in more detail herein below.

The term “antibody” refers to any immunoglobulin and encompasses any polypeptide comprising an antigen-binding site. The term includes, but is not limited to, polyclonal, monoclonal, monospecific, polyspecific, non-specific, humanized, human, single-chain, chimeric, synthetic, recombinant, hybrid, mutated, grafted, and in vitro generated antibodies.

In further embodiments of the invention, the product is an antibody that has a CH 2/CH 3 region and therefore is amenable to purification by Protein A chromatography. The term “CH 2/CH 3 region” refers to those amino acid residues in the Fc region of an immunoglobulin molecule that interact with Protein A. In particular, the antibody of the invention is a monoclonal Fc-containing antibody capable of binding to a protein A ligand.

Examples of Fc-containing monoclonal antibodies of the invention includes the below described antibodies binding to Calcitonin Gene Related Peptide (CGRP), Pituitary Adenylate Cyclase-Activating Polypeptide (PACAP) and Adrenocorticotropic Hormone (ACTH).

Calcitonin Gene Related Peptide (CGRP) is produced as a multifunctional neuropeptide of 37 amino acids in length. Two forms of CGRP, the CGRP-alpha and CGRP-beta forms, exist in humans and have similar activities. CGRP-alpha and CGRP-beta differ by three amino acids in humans, and are derived from different genes. The CGRP family of peptides includes amylin, adrenomedullin, and calcitonin, although each has distinct receptors and biological activities. Doods, H., Curr. Op. Invest. Drugs, 2 (9): 1261-68 (2001).

Migraines are neurovascular disorder affecting approximately 10% of the adult population in the U.S., and are typically accompanied by intense headaches. Approximately 20-30% of migraine sufferers experience aura, comprising focal neurological phenomena that precede and/or accompany the event. CGRP is believed to play a prominent role in the development of migraines. For example, plasma concentrations of CGRP were identified elevated in jugular venous blood during the headache phase of migraines, to the exclusion of other neuropeptides. Moreover, according to Arulmozhi et al, (2005) the following has been identified in migraine sufferers: (1) a strong correlation between plasma CGRP concentrations and migraines; (2) the infusion of CGRP produced a migraine-like headache; (3) baseline CGRP levels were elevated; and (4) changes in plasma CGRP levels during migraine attacks significantly correlated with headache intensity. (Arulmozhi, D. K., et al., Vas. Pharma., 43:176-187 (2005)).

In certain embodiments the invention relates to the CGRP binding antibody Eptinezumab with the following sequences

Heavy Chain CDRs for Eptinezumab

SEQ ID No.: 1
CDR-H1: GYYMN
SEQ ID No.: 2
CDR-H2: VIGINGATYYASWAKG
SEQ ID No.: 3
CDR-H3: GDI

The Variable Heavy Chain of Eptinezumab Comprises

SEQ ID No.: 4
EVOLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSS

The Heavy Chain of Eptinezumab comprises

SEQ ID No.: 5
EVQLVESGGG LVQPGGSLRL SCAVSGIDLS GYYMNWVRQA
PGKGLEWVGV IGINGATYYA SWAKGRFTIS RDNSKTTVYL
QMNSLRAEDT AVYFCARGDI WGQGTLVTVS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDARVE PKSCDKTHTC PPCPAPELLG GPSVFLFPPK
PKDTLMISRT PEVTCVVVDV SHEDPEVKFN WYVDGVEVHN
AKTKPREEQY ASTYRVVSVL TVLHQDWLNG KEYKCKVSNK
ALPAPIEKTI SKAKGQPREP QVYTLPPSRE EMTKNQVSLT
CLVKGFYPSD IAVEWESNGQ PENNYKTTPP VLDSDGSFFL
YSKLTVDKSR WQQGNVFSCS VMHEALHNHY TQKSLSLSPG

A C-terminal lysine (K) may be present in case the Heavy Chain is not processed in a system that cleave this, e.g. in a yeast such as Pichia, but the c-terminal lysine will usually be cleaved in a CHO expression system:

SEQ ID No.: 6
EVQLVESGGG LVQPGGSLRL SCAVSGIDLS GYYMNWVRQA
PGKGLEWVGV IGINGATYYA SWAKGRFTIS RDNSKTTVYL
QMNSLRAEDT AVYFCARGDI WGQGTLVTVS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDARVE PKSCDKTHTC PPCPAPELLG GPSVFLFPPK
PKDTLMISRT PEVTCVVVDV SHEDPEVKFN WYVDGVEVHN
AKTKPREEQY ASTYRVVSVL TVLHQDWLNG KEYKCKVSNK
ALPAPIEKTI SKAKGQPREP QVYTLPPSRE EMTKNQVSLT
CLVKGFYPSD IAVEWESNGQ PENNYKTTPP VLDSDGSFFL
YSKLTVDKSR WQQGNVFSCS VMHEALHNHY TQKSLSLSPGK

Light Chain CDRs for Eptinezumab

SEQ ID No.: 7
CDR-L1: QASQSVYHNTYLA
SEQ ID No.: 8
CDR-L2: DASTLAS
SEQ ID No.: 9
CDR-L3: LGSYDCTNGDCFV

The Variable Light Chain of Eptinezumab comprises

SEQ ID No.: 10
QVLTQSPSSLSASVGDRVTINCQASQSVYHNTYLAWYQQKPGKVPKQLI
YDASTLASGVPSRFSGSGSGTDFTLTISSLQPEDVATYYCLGSYDCTNG
DCFVFGGGTKVEIKR

The Light Chain of Eptinezumab comprises

SEQ ID No.: 11
QVLTQSPSSL SASVGDRVTI NCQASQSVYH NTYLAWYQQK
PGKVPKQLIY DASTLASGVP SRFSGSGSGT DFTLTISSLQ
PEDVATYYCL GSYDCTNGDC FVFGGGTKVE IKRTVAAPSV
FIFPPSDEQL KSGTASWVCL LNNFYPREAK VQWKVDNALQ
SGNSQESVTE QDSKDSTYSL SSTLTLSKAD YEKHKVYACE
VTHQGLSSPV TKSFNRGEC

Pituitary Adenylate Cyclase-Activating Polypeptide (“PACAP”) is a member of the secretin/vasoactive intestinal peptide (“VIP”)/growth hormone-releasing hormone (“GHRH”) family. PACAP is a multifunctional vasodilatory peptide that exists in two α-amidated active forms, one with 38 amino acids and the other with 27 amino acids. Both peptides have the same N-terminal 27 amino acids and are synthesized from the same precursor protein, preproPACAP (See, Moody et al., Curr. Opin. Endocrinol. Diabetes Obes., 18 (1): 61-67, 2011). PACAP38 is the more prevalent active form, representing up to 90% of PACAP forms in mammalian tissues (See, Kaiser and Russo, Neuropeptides, 47:451-461, 2013). The sequence of PACAP38 is identical in all mammals and differs from the avian and amphibian orthologs by only one amino acid (See, Vaudry et al., Pharmacol. Rev., 52:269-324, 2000). The secretin/VIP/GHRH family includes mammalian peptide histidine methionine (“PHM”), secretin, glucagon, glucagon-like peptide-1 (“GLP1”), glucagon-like peptide-2 (“GLP2”), glucose-dependent-insulinotropic-polypeptide (“GIP”), and growth-hormone-releasing-factor (“GRF”). PACAP27 has 68% sequence identity to VIP at the amino acid level (See, Vaudry et al., 2000).

PACAP is hypothesized to play a role in a multitude of diseases and disorders, including but not limited to migraine, headache, and pain, though such a role for PACAP has not been clinically demonstrated. Migraines are believed to have a neurovascular component. Migraines affect approximately 10% of the adult population in the U.S. and are typically accompanied by intense headaches. Approximately 20-30% of migraine sufferers experience aura, comprising focal neurological phenomena that precede and/or accompany the event. A role for PACAP in migraine has been suggested by several observations: (1) plasma levels of PACAP are elevated during migraine attacks (ictal), as compared to interictal levels, in humans (see Tuka et al., Cephalalgia, 33 (13): 1085-1095, 2013); (2) an infusion of PACAP38 triggered headaches in healthy subjects, and headaches followed by migraine-like attacks in migraineurs (see Schytz et al., Brain, 132:16-25, 2009; and Amin et al., Brain, 137:779-794, 2014, respectively); (3) PACAP-induced vasodilation may play a role in neurogenic inflammation (see Kaiser and Russo, Neuropeptides, 47:451-461, 2013); and (4) PACAP-induced migraines are associated with photophobia, phonophobia, nausea, and respond to triptans (see Amin et al., Brain, 32:140-149, 2012). PACAP has also been shown to induce vasodilation, photophobia, as well as mast cell degranulation and neuronal activation (See, Markovics et al., Neurobiology of Disease, 45:633-644, 2012; Baun et al., Cephalalgia, 32 (4):337-345, 2012; Chan et al., Pharmacology & Therapeutics, 129:332-351, 2011).

In certain embodiments the invention relates to an antibody binding to PACAP (named PACAP-1) with the following sequences

Heavy Chain CDRs for PACAP-1

CDR-H1:
SEQ ID No.: 12
SYYMT
CDR-H2:
SEQ ID No.: 13
FIDAGGDAYYASWAKG
CDR-H3:
SEQ ID No.: 14
DLDL

The Variable Heavy Chain of PACAP-1 Comprises

SEQ ID No.: 15
EVQLVESGGGLVQPGGSLRLSCAASGIDLNSYYMTWVRQAPGKGLEWIG
FIDAGGDAYYASWAKGRFTISRDNSKNTVYLQMNSLRAEDTAVYFCARD
LDLWGQGTLVTVSS

The Heavy Chain of PACAP-1 comprises

SEQ ID No.: 16
EVQLVESGGGLVQPGGSLRLSCAASGIDLNSYYMTWVRQAPGKGLEWIG
FIDAGGDAYYASWAKGRFTISRDNSKNTVYLQMNSLRAEDTAVYFCARD
LDLWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDT
LMISRTPEVTCVWVDVSHEDPEVKFNWYVDGVEVHNAKTKPREEQYAST
YRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

Light Chain CDRs of PACAP-1

CDR-L1:
SEQ ID No.: 17
QSSESVYGNYLA
CDR-L2:
SEQ ID No.: 18
EASKLES
CDR-L3:
SEQ ID No.: 19
AGGDISEGVA

The Variable Light Chain of PACAP-1 comprises

SEQ ID No.: 20
DIQLTQSPSTLSASVGDRVTITCQSSESVYGNYLAWFQQKPGKAPKFLI
YEASKLESGVPSRFSGSGSGTEFTLTISSLQPDDFATYYCAGGDISEGV
AFGGGTKVEIKR

The Light Chain of PACAP-1 comprises

SEQ ID No.: 21
DIQLTQSPSTLSASVGDRVTITCQSSESVYGNYLAWFQQKPGKAPKFLI
YEASKLESGVPSRFSGSGSGTEFTLTISSLQPDDFATYYCAGGDISEGV
AFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

POMC peptides, including ACTH (Adrenocorticotropic Hormone), are believed to act primarily through melanocortin receptors (MCRs), a family of five G protein-coupled receptors (i.e., MC1R, MC2R, MC3R, MC4R and MC5R). MCRs are expressed in diverse tissues, and serve discrete physiological functions. MC1R, which is expressed on melanocytes, macrophages and adipocytes, is involved in pigmentation and inflammation. MC2R, which is expressed in the adrenal cortex, is involved in adrenal steroidogenesis. MC3R, which is expressed in the central nervous system (CNS), gastrointestinal (GI) tract and kidney, is involved in energy homeostasis and inflammation. MC4R, which is expressed in the CNS and spinal cord, is involved in energy homeostasis, appetite regulation and erectile function. MC5R, which is expressed on lymphocytes and exocrine cells, is involved in exocrine function and regulation of sebaceous glands. See Ramachandrappa et al., Frontiers in Endocrinology 4:19 (2013).

ACTH, one of the major end-products of POMC processing, is a hormone that is essential for normal steroidogenesis and the maintenance of normal adrenal weight. ACTH is secreted by the pituitary gland in response to physiological or psychological stress and its principal effect is increased production and release of corticosteroids. In particular, ACTH is secreted from corticotropes in the anterior lobe (or adenohypophysis) of the pituitary gland in response to the release of the hormone corticotropin-releasing hormone (CH) by the hypothalamus. Once secreted, ACTH then travels to the adrenal cortex, where it binds to and activates MC2R. Activation of MC2R results in the production of CAMP in the adrenal cell. cAMP binds and activates protein kinase (PICA), which activates the conversion of the lipid cholesterol to the steroid hormone Cortisol.

Cortisol is a hormone that affects numerous biological processes in order to restore homeostasis after stress. Exemplary processes regulated by Cortisol include regulating glucose homeostasis, increasing blood pressure, gluconeogenesis, promoting metabolism of glycogen, lipids, and proteins, and suppressing the immune system. Under normal physiological conditions, Cortisol levels are tightly regulated. However, in some conditions (including diseases and disorders further described herein), Cortisol levels are elevated. The overproduction of Cortisol has been shown to have many negative effects, such as damaging the hippocampus, a region of the brain that is critical for cognitive functions and regulation of the hypothalamus pituitary adrenal axis; increasing fat deposits, blood pressure levels, and blood sugar levels; bone loss; muscle weakness; and suppression of the immune system. Therefore, elevated Cortisol levels may play a role in ACTH-driven hypercortisolism (such as Cushing's Disease or Cushing's Syndrome), obesity, diabetes, sleep apnea, depression, anxiety disorders, cancer (such as Cushing's Syndrome resulting from ectopic ACTH expression, e.g., in small cell lung cancer, non-small cell lung cancer (NSCLC), pancreatic carcinoma, neural tumors, or thymoma), muscle atrophies, hypertension, cognitive dysfunction, galactorrhea and metabolic syndromes.

Aldosterone is a hormone released by the adrenal glands that helps regulate blood pressure. In particular, aldosterone increases the reabsorption of sodium and water and the release of potassium in the kidneys. In some disease conditions, aldosterone levels are elevated. For example, primary and secondary hyperaldosteronism occur when the adrenal gland releases too much of the hormone aldosterone. Primary hyperaldosteronism such as Conn's syndrome results from a problem with the adrenal gland itself that causes the release of too much aldosterone, whereas the excess aldosterone in secondary hyperaldosteronism is caused by something outside the adrenal gland that mimics the primary condition, e.g., by causing the adrenal gland to release too much aldosterone. Primary hyperaldosteronism used to be considered a rare condition, but some experts believe that it may be the cause of high blood pressure in some patients. Most cases of primary hyperaldosteronism

In certain embodiments the invention relates to an antibody binding to ACTH (named ACTH-1) with the following sequences

Heavy Chain CDRs for ACTH-1

CDR-H1:
SEQ ID No.: 22
SGYDIC
CDR-H2:
SEQ ID No.: 23
CIDTGSGNTYYASSAKG
CDR-H3:
SEQ ID No.: 24
GISSI

The Variable Heavy Chain of ACTH-1 Comprises

SEQ ID No.: 25
EVQLVESGGGLVQPGGSLRLSCAASGFTVSSGYDICWVRQAPGKGLEWI
GCIDTGSGNTYYASSAKGRFTMSRDNSKNTVYLQMNSLRAEDTAVYYCA
KGISSIWGQGTLVTVSS

The Heavy Chain of ACTH-1 comprises

SEQ ID No.: 26
EVQLVESGGGLVQPGGSLRLSCAASGFTVSSGYDICWVRQAPGKGLEWI
GCIDTGSGNTYYASSAKGRFTMSRDNSKNTVYLQMNSLRAEDTAVYYCA
KGISSIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
ASTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPG

The Heavy Chain of ACTH-1 may comprise a terminal Lysine (K) if expressed in yeast (e.g. Pichia pastoris cells)

SEQ ID No.: 32
EVQLVESGGGLVQPGGSLRLSCAASGFTVSSGYDICWVRQAPGKGLEWI
GCIDTGSGNTYYASSAKGRFTMSRDNSKNTVYLQMNSLRAEDTAVYYCA
KGISSIWGQGTLVTVSSASTKGPSVFPLAPSSKSTSGGTAALGCLVKDY
FPEPVTVSWNSGALTSGVHTFPAVLQSSGLYSLSSWVTVPSSSLGTQTY
ICNVNHKPSNTKVDKKVEPKSCDKTHTCPPCPAPELLGGPSVFLFPPKP
KDTLMISRTPEVTCVVDVSHEDPEVKFNWYVDGVEVHNAKTKPREEQYA
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGK

Light Chain CDRs for ACTH-1

CDR-L1:
SEQ ID No.: 27
QASQTISSDLA
CDR-L2:
SEQ ID No.: 28
AASKLTS
CDR-L3:
SEQ ID No.: 29
QTYYDIIDDGAT

The Variable Light Chain of ACTH-1 comprises

SEQ ID No.: 30
DIQMTQSPSTLSASVGDRVTITCQASQTISSDLAWYQQKPGKAPKLLIY
AASKLTSGVPSRFSGSGSGTEFTLTISSLQPDDFATYYCQTYYDIIDDG
ATFGGGTKVEIKR

The Light Chain of ACTH-1 comprises

SEQ ID No.: 31
DIQMTQSPSTLSASVGDRVTITCQASQTISSDLAWYQQKPGKAPKLLIY
AASKLTSGVPSRFSGSGSGTEFTLTISSLQPDDFATYYCQTYYDIIDDG
ATFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC

The antibody preparations used with methods described herein can be from a number of sources including, but not limited to, serum of an immunized animal, ascites fluid, hybridoma or myeloma supernatants, conditioned culture medium derived from culturing a recombinant cell line that expresses the antibody molecule, or from a cell extract of antibody-producing cells. In one embodiment of the invention, the product is an antibody from conditioned culture medium of an antibody-producing recombinant cell line. Although there can be some variation from cell line to cell line and among the various antibody products, based on the disclosure herein, it is well within the purview of one of ordinary skill in this art to adapt the invention herein to a particular combination of antibody protein and producing cell line.

In certain embodiments, at least one impurity is in the fluid load applied to the chromatographic column, and at least one wash solution containing arginine or an arginine salt or arginine derivative is used to remove the impurity that is bound to the resin of said chromatographic column. In one embodiment of the invention, the purified product contains less than 60% impurities (e.g., host cell proteins), in one embodiment about 40% impurities, in one embodiment about 20% impurities, in one embodiment about 10% impurities, in one embodiment about 5% impurities, in one embodiment less than 3% impurities, and in another embodiment less than 1% impurities after the wash and elution of the chromatographic column. Impurities include, but are not limited to, undesirable protein variants, such as aggregated proteins, high molecular weight species, low molecular weight species and fragments, and deamidated species; other proteins from host cells that secrete the protein being purified; host cell DNA; components from the cell culture medium, molecules that are part of an absorbent used for affinity chromatography that leach into a sample during prior purification steps, for example, Protein A and Protein G; an endotoxin; a nucleic acid; a virus, or a fragment of any of the forgoing.

The chromatographic column used in a method described herein is, for example, an affinity chromatography column, a hydrophobic interaction chromatography column, an immobilized metal affinity chromatography column, a size exclusion chromatography column, a diafiltration, ultrafiltration, viral removal filtration, and/or ion exchange chromatography column, a Protein A chromatography column or a Protein G chromatography column. A Protein A chromatography column can be, for example, PROSEP-A™ (Millipore, U.K.), Protein A Sepharose FAST FLOW™ (GE Healthcare, Piscataway, N.J.), TOYOPEARL™ 650 M Protein A (TosoHass Co., Philadelphia, Pa.), or MabSelect™ column (GE Healthcare, Piscataway, N.J.) or MabSelect SuRe (AKTA Avant).

Before contacting the resin in a chromatographic column with a load fluid, it may be necessary to adjust parameters such as pH, ionic strength, and temperature and in some instances the addition of substances of different kinds. Thus, it is an optional step to perform an equilibration of the chromatographic column by washing it with a solution (e.g., a buffer for adjusting pH, ionic strength, etc., or for the introduction of a detergent) bringing the necessary characteristics for binding and purification of the product.

In one embodiment of the invention, a Protein A chromatographic column is equilibrated and washed with a wash solution, thereby bringing the necessary characteristics for purifying the product. In one embodiment of the invention, the Protein A chromatographic column may be equilibrated using a solution containing a salt, e.g., about 10 mM to about 30 mM NaPO4, and about 100 mM to about 150 mM NaCl. The pH of the equilibration buffer may range from about 6.0 to about 7.0. In one embodiment, the pH of the equilibration buffer is about 6.8. After contacting the resin in a chromatographic column (e.g., a Protein A chromatographic column) with the load fluid, the resin is first washed. A first wash may according to one embodiment of the invention be with the above-mentioned equilibrium solution.

In accordance with the invention, following the equilibrium of the Protein A chromatographic column, and optionally a first wash without arginine or arginine salt or arginine derivative, a subsequent wash solution used in the method described herein may contain arginine or an arginine derivative. The arginine derivative can be, but is not limited to, acetyl arginine, agrnatine, arginic acid, N-alpha-butyroyl-L-arginine, or N-alpha-pyvaloyl arginine.

The concentration of arginine or arginine derivative in the wash solution is between about 500 mM and about 600 mM, e.g., 500 mM, 525 mM, 550 mM, 575 mM or 600 mM. In certain embodiments, the concentration of arginine or arginine derivative in the wash solution is between about 500 mM to about 600 mM or about 525 mM to about 575 mM, or about 550 mM to about 575 mM. In certain embodiments, the concentration of arginine or arginine derivative in the wash solution is greater than about 500 mM and less than about 600 mM.

The pH of the wash solution is generally between about 8.0 and about 8.7, for example, 8.1, 8.2, 8.3,.8.4, 8.5, 8.6, and 8.7. In some cases, the pH of the wash solution is greater than 8.0 and less than about 8.7. The wash solution may contain 20 mM to 50 mM sodium phosphate (Na₂HPO₄) (e.g., 20 mM, 30 mM, 40 mM or 50 mM). In one embodiment the bound medium is washed with 5 column volumes of the wash solution, followed by an elution step.

The wash step with arginine or arginine derivative, may subsequently be followed by one or more wash steps without arginine or arginine derivative. According to one embodiment this may be using a Sodium Acetate containing wash medium (e.g. at a sodium acetate concentration of about 20 mM to about 50 mM, such at 20 mM, 30 mM 40 mM or 50 mM) at a pH about 5 to about 6 (e.g. pH about 5.4)

In certain embodiments of the invention, the product may be eluted from a washed resin from e.g., a Protein A chromatographic column. To elute a product from a Protein A chromatographic column, the washed resin from said chromatographic column is contacted with an elution buffer. In some embodiments, the elution buffer contains about 15 mM to about 50 mM Acetic Acid. In further embodiments, the elution buffer may also contain 20 mM to 50 mM glycine. The pH of the elution buffer may range from about 2.0 to about 5.0. In one embodiment, the pH of the elution buffer is about 4.0. In another embodiment the pH of the buffer is about 3.65 or between 3.55 to 3.57.

The resin from the chromatographic column may optionally be cleaned, i.e., stripped and regenerated, after elution of the antibody. This procedure is typically performed regularly to minimize the building up of impurities on the surface of the solid phase and/or to sterilize the matrix to avoid contamination of the product with microorganisms.

Buffer components may be adjusted according to the knowledge of the person of ordinary skill in the art. Sample buffer composition ranges are provided in the Examples below. Not all of the buffers or steps are necessary but are provided for illustration only. A high throughput screen, as described in the Examples, may be used to efficiently optimize buffer conditions for Protein A column chromatography.

The eluate can include a product and the ratio of the product to host cell protein is increased compared to a corresponding method in which no detectable amount of arginine or arginine derivative is used in a wash solution.

The present invention also relates to a product prepared according to a method described herein. In general, it will typically be desirable to further isolate and/or purify products isolated according to the present invention and formulate them for pharmaceutical use according to standard methods. For proteins, see for example Protein Purification Principles and Practice 2nd Edition, Springer-Verlag, New York, 1987; Higgins, S. J. and Hames, B. D. (eds.), and Deutscher, M. P., Simon, M. I., Abelson, J. N. (eds.), Guide to Protein Purification: Methods in Enzymology (Methods in Enzymology Series, Vol 182), Academic Press, 1997, incorporated herein by reference. One of ordinary skill in the art will appreciate that the exact techniques used will vary depending on the character of the product. Products of the invention having pharmacologic activity will be useful in the preparation of pharmaceuticals. These may be administered to a subject or may first be formulated for delivery by any available route including, but not limited to parenteral (e.g., intravenous), intradermal, subcutaneous, oral, nasal, bronchial, ophthalmic, transdermal (topical), transmucosal, rectal, and vaginal.

A pharmaceutical composition of the product is formulated to be compatible with its intended route of administration according to methods known in the art, see for example, Remington: The Science & Practice of Pharmacy”, 19th ed., Williams & Williams, (1995), and the “Physician's Desk Reference”, 52nd ed., Medical Economics, Montvale, N.J. (1998). In some embodiments, the product is formulated using sterile water (e.g., SWFI), buffered saline (e.g., phosphate buffered saline), polyol (e.g., glycerol, propylene glycol, liquid polyethylene glycol), or suitable mixtures thereof.

Non-limiting examples of products that can be recovered using the methods described herein include a protein or a peptide, e.g., an antibody, an antibody fragment, a recombinant protein, a naturally secreted protein, a protein or a peptide that is engineered to be secreted, a non-protein product that is produced by a cell, or a combination of the foregoing products.

### EMBODIMENTS

The present invention further relates to the following embodiments (E)

E1. A method for reducing impurities in an eluate comprising a product, the method comprising:
(a) providing a load fluid comprising a product and one or more impurities, wherein the product is an Fc-containing monoclonal antibody,
(b) applying the load fluid in a) to a Protein A chromatography column under conditions suitable for binding the product,
(c) optionally contacting the Protein A chromatography column with a first wash solution,
(d) applying one or more wash solutions (such as 1, 2 or 3 wash solutions) to the Protein A chromatography column, wherein the wash solution comprises arginine or an arginine salt or other arginine derivative in a concentration of above 500 mM, and wherein the pH of the wash solution is greater than 8.0,
(e) optionally contacting the Protein A chromatography column in d) with another second wash solution,
(f) contacting the washed Protein A chromatography column in d) or e) with an elution solution under conditions suitable for eluting the product, and
(g) collecting an eluate comprising the product.

E2. The method of Embodiment 1, wherein the load fluid in step (a) is obtained from a CHO cell expressing Eptinezumab, PACAP-1, or ACTH-1, such as the conditioned culture medium from the CHO cells expressing said antibodies.

E3. The method of Embodiment 1, wherein the impurities in step (a) is selected from the group comprising DNA, RNA, endotoxins, proteins (other than Eptinezumab, PACAP-1, or ACTH-1) and Host Cell Proteins (such as undesired protein from the CHO cells).

E4. The method of Embodiment 1, wherein the concentration of the arginine or the arginine salt or arginine derivative is in the range of:
(I) about 500 mM to about 600 mM;
(II) about 525 mM to about 575 mM,
(III) about 550 mM to about 575 mM or
(IV) is about 575 mM.

E5. The method off Embodiments 1 or 2, wherein the pH of the one or more wash solutions of (d):
(i) range from about 8.0 to about 8.7;
(ii) range from about 8.2 to about 8.7;
(iii) range about 8.5 to about 8.7;
(iv) is/are about 8.5;
(v) is/are about 8.6; or
(vi) is/are about 8.7.

E6. The method of Embodiments 1-5, wherein the arginine salt or arginine derivative comprises Arg·HCl, acetyl arginine, agmatine, arginic acid, N-alpha-butyroyl-L-arginine, or N-alpha-pyvaloyl arginine.

E7. The method of any one of Embodiments 1-6, wherein the monoclonal antibody is specific for CGRP, such as Eptinezumab as defined by
Variable Heavy Chain Seq ID NO.: 4 and Variable Light Chain Seq ID No.: 10 and/or
Heavy Chain Seq ID Nos.: 5 or 6 and Light Chain Seq ID No.: 11

E8. The method of any one of Embodiments 1-6, wherein the monoclonal antibody is specific for PACAP, such as PACAP-1 as defined by
Variable Heavy Chain Seq ID NO.: 15 and Variable Light Chain Seq ID No.: 20 and/or
Heavy Chain Seq ID No.: 16 and Light Chain Seq ID No.: 21

E9. The method of any one of Embodiments 1-6, wherein the monoclonal antibody is specific for ACTH, such as ACTH-1 as defined by
Variable Heavy Chain Seq ID NO.: 25 and Variable Light Chain Seq ID No.: 30 and/or
Heavy Chain Seq ID No.: 26 or Heavy Chain Seq ID No.: 32 and Light Chain Seq ID No.: 31

E10. The method of any one of Embodiments 1-9, wherein the concentration of arginine in the wash solution in d) is about 575 mM and PH is 8.5 or 8.7.

E11. The method of any one of Embodiments 1-9, wherein the concentration of arginine in the one or more wash solutions in d) is about 525 mM and the pH is 8.5 or 8.7.

E12. The method of any one of Embodiments 1-11, wherein one or more of the impurities are a host cell protein, a nucleic acid, a product variant, and/or an endotoxin.

E13. The method of any one of Embodiments 1-12, wherein the elution solution comprises about 15 mM to about 50 mM Acetic Acid and optionally comprises about 20 mM to 50 mM glycine.

E14. The method of any one of Embodiments 1-13, wherein the elution solution has a pH of between about 2 and about 5.

E15. The method of any one of Embodiments 1-14, wherein the elution solution has a pH of about 4.

E16. The method of any one of Embodiments 1-15, wherein the one or more wash solutions in c) and e) do not contain arginine or an arginine salt or arginine derivative.

E17. A pharmaceutical composition comprising Eptinezumab, PACAP-1 or ACTH-1 obtained using the method according to Embodiment 1.

E18. Use of the pharmaceutical composition according to Embidiment 17 comprising Eptinezumab, PACAP-1 or ACTH-1 as a medicament.

E19. The pharmaceutical composition comprising Eptinezumab according to Embodiment E18 for use in the treatment of headache, migraine (such as chronic or episodic migraine) and cluster headache.

E20. The pharmaceutical composition comprising ACTH-1 according to Embodiment 17 for use in the treatment of Cushings Disease or Congenital Adrenal Hyperplasia.

E21. The pharmaceutical composition comprising PACAP-1 according to Embodiment 17 for use in the treatment of headache, migraine (such as chronic or episodic migraine) and cluster headache.

E22. A method for treating headache, migraine (such as chronic or episodic migraine) and cluster headache comprising administering a terapeutical effective amount of Eptinezumab or PACAP-1 obtained by the method of Embodiment 1.

E23. A method for treating Cushings Disease or Congenital Adrenal Hyperplasia comprising administering a terapeutical effective amount of ACTH-1 obtained by the method of Embodiment 1.

### EXAMPLES

### Example 1

To explore the effect of different arginine concentrations and pH levels in a wash solution on a Protein A column, the inventors of the present invention tested different pH and arginine conditions and measured different impurities, including the HCP concentrations.

Ranges tested was pH 7.6-8.7, Arginine 375-575 mM and the Length of Wash varied between 5-10 column volumes (CVs).

The Protein A column was loaded with filtered clarified harvested media from CHO cells expressing Eptinezumab. Eptinezumab were harvested from 6×3L bioreactors, and depth filtered harvest at 4.34 g/L.

The protein A column was run under the following conditions:

| | | Volume | Linear Velocity | Limits/Ranges/ |
|---|---|---|---|---|
| Step | Buffer | (CV) | (cm/hr) | Targets/Note(s) |
| Pre-Sanitization | 0.2M NaOH | 3 | 250 | Contact Time = 15 mg |
| Equilibration | 20 mM Sodium Phosphate, | 5 | 250 | pH: 6.8 ± 0.2 @ 24-26° C. |
| | 100 mM NaCl, pH 6.8 | | | Conductivity: 16.5-19.0 mS/cm @ |
| | | | | 24-26° C. |
| Load | Filtered Clarified | Variable | ≤150 | Loading density target: 40 g/L |
| | Harvest | | | Residence time: 8 min |
| | | | | Load ID: CP57-E331 |
| Wash 1 | 20 mM Sodium Phosphate, | 5 | 250 | Same as Equilibration |
| | 100 mM NaCl, pH 6.8 | | | |
| Wash 2 | Variable Arg 375 to 575 mM | Variable5-10CV | 250 | Variable for this Study |
| | Variable pH 7.6 to 8.5 plus and 8.7 | | | |
| Wash 3 | 25 mM Sodium Acetate pH 5.4 | 5 | 250 | Different from EQ |
| Eluate Conditioning | None | NA | | |
| Elution | 50 mM Acetate, 50 mM | 5 | 250 | pH: 3.6 ± 0.2 @ 24-26° C. |
| | Glycine, pH 3.6 | | | Conductivity: 0.5-1.6 mS/cm @ |
| | | | | 24-26° C. |
| | | | | Collection Start: 0.25 AU/cm |
| | | | | Collection End: 0.25 AU/cm |
| Strip | 100 mM Acetic Acid | 3 | 250 | |
| Rinse | 20 mM Sodium Phosphate, | 3 | 250 | Same as Equilibration |
| | 100 mM NaCl, pH 6.8 | | | |

The Protein A Column used was MabSelect SuRE resin and the instrument AKTA Avant

(Column Name: 20.0 cm/10ml/0.8 cm—MabSelect SuRe Lx 10253604—Col #817)

The measurements of HCP were performed as per protocol:
Immunoenzymetric Assay for the Measurement of CHO Host Cell Proteins (Cygnus Technologies Catalog #550-1)
   Reagents & Materials
   Anti-CHO: HRP (F551-1) Affinity purified goat antibody conjugated to HRP in a protein matrix with preservative. 1×12 mL
   Anti-CHO coated microtiter strips F552-1* 12×8 well strips in a bag with desiccant
   CHO HCP Standards F553-1 CHO HCPs in bovine serum albumin with preservative. Standards at 0, 1, 3, 12, 40, and 100 ng/ml. 1 mL/vial
   Stop Solution 0.5 M sulfuric acid. 1×12 mL
   TMB Substrate F005 3,3′,5,5′ Tetramethylbenzidine. 1×12 mL
   Wash Concentrate (20X) F004 Tris buffered saline with preservative. 1×50 mL
Measurements of-Host Cell Protein (HCP)
Assay Protocol.
1. Pipette 100 μL of anti-CHO: HRP (#F551-1) into each well.
2. Pipette 50 μL of standards, controls and samples into wells indicated on work list.
3. Cover & incubate on orbital shaker at 400-600 rpm for 2 hours at room temperature, 24° C.+4° C. 4. Dump contents of wells into waste. Blot and gently but firmly tap over absorbent paper to remove most of the residual liquid. Overly aggressive banging of the plate or use of vacuum aspiration devices in an attempt to remove all residual liquid is not necessary and may cause variable dissociation of antibody bound material resulting in lower ODs and worse precision. Fill wells generously to overflowing with diluted wash solution using a squirt bottle or by pipetting in ˜350 μL. Dump and tap again. Repeat for a total of 4 washes. Wipe off any liquid from the bottom outside of the microtiter wells as any residue can interfere in the reading step. Do not allow wash solution to remain in wells for longer than a few seconds.
5. Pipette 100 μL of TMB substrate (#F005).
6. Incubate at room temperature for 30 minutes.
7. Pipette 100 μL of Stop Solution (#F006).
8. Read absorbance at 450/650 nm.

Table 1 shows the wash conditions, the yield and the HCP content in the eluent for the wash experiments

| Run | W2 | W2 Arg | W2 | Actual | Pool Volume (mL) | Pool Conc. | % | HCP |
|---|---|---|---|---|---|---|---|---|
| Order | pH | (mM) | CV | Buffer pH | Calc. by weight | (mg/mL) | Yield | (ppm) |
| 1 | 8.5 | 375 | 7.5 | 8.51 | 15.3 | 26.6 | 101.7 | 1183.3 |
| 2 | 8.05 | 375 | 10 | 8.02 | 16.1 | 25.3 | 101.8 | 1214 |
| 3 | 7.6 | 575 | 5 | 7.59 | 16.4 | 25 | 102.5 | 1456.2 |
| 4 | 8.5 | 475 | 10 | 8.5 | 15.8 | 25.2 | 99.5 | 813.7 |
| 5 | 8.05 | 475 | 7.5 | 7.97 | 16.2 | 25 | 101.3 | 1143.4 |
| 6 | 8.05 | 475 | 7.5 | 7.97 | 16.6 | 24.4 | 101.3 | 1250.7 |
| 7 | 8.05 | 575 | 10 | 8.03 | 13.1 | 30.7 | 100.5 | 880 |
| 8 | 8.05 | 575 | 10 | 8.03 | 14.8 | 27.1 | 100.3 | 788.4 |
| 9 | 8.05 | 475 | 7.5 | 7.97 | 15.7 | 26 | 102.1 | 1201.9 |
| 10 | 8.5 | 575 | 7.5 | 8.51 | 13.7 | 26.6 | 91.1 | 721.9 |
| 11 | 7.6 | 375 | 5 | 7.6 | 16 | 25.6 | 102.4 | 3647 |
| 12 | 8.5 | 575 | 7.5 | 8.51 | 15.4 | 26.4 | 101.6 | 746.5 |
| 13 | 7.6 | 433 | 10 | 7.58 | 15.1 | 26.8 | 101.2 | 1375.1 |
| 14 | 8.5 | 475 | 5 | 8.5 | 16.1 | 25.4 | 102.2 | 1227.7 |
| 15 | 8.5 | 475 | 5 | 8.5 | 16.2 | 25 | 101.3 | 1069.6 |
| 16 | 7.9 | 500 | 5 | 7.9 | 15.9 | 25.6 | 101.8 | 1026.6 |
| 17 | 7.9 | 450 | 5 | 7.9 | 16.6 | 24.7 | 102.5 | 1565.5 |
| 18 | 7.9 | 450 | 10 | 7.9 | 16.5 | 24.7 | 101.9 | 963.1 |
| 19 | 8.7 | 575 | 5 | 8.74 | 16.5 | 24.6 | 101.5 | 865.7 |
| 20 | 8.5 | 475 | 5 | 8.5 | 17.5 | 23.5 | 102.8 | 898.5 |
| 21 | 8.5 | 375 | 10 | 8.51 | 17.6 | 23.2 | 102.1 | 800.5 |
| 22 | 8.5 | 575 | 7.5 | 8.51 | 18 | 22.7 | 102.2 | 666.2 |

Similar results have been obtained by purifying the PACAP and ACTH antibodies (Named PACAP-1 and ACTH-1 in the present application) as described in the present invention. HCP content for PACAP sample ranged from 156.9 to 294.7 following purification under same conditions as above, whereas for ACTH an HCP content of about 245 was obtained running at pH about 8.5 and Arginine concentration of about 575 mM.

In a further study using ph 8.5 and arginine at 575 mM the HCP content was reduced to from about 307684 ng HCP/mg protein and 700688 ng HCP/mg protein to about 224 ng HCP/mg protein and 220 ng HCP/mg protein respectively in two separate runs for PACAP-1. In addition, ACTH-1 purifications show a reduction in HCP ng/mg protein from 891432 to 326 ng/mg (or 2104671 ng/ml HCP to 10002 ng/mL HCP) and 10042454 ng/mg to 232 ng/mg (or 2435063 ng/mL HCP to 7062 ng/ml).

1. A method for reducing impurities in an eluate comprising a product, the method comprising:
(a) providing a load fluid comprising a product and one or more impurities, wherein the product is an Fc-containing monoclonal antibody,
(b) applying the load fluid in a) to a Protein A chromatography column under conditions suitable for binding the product,
(c) optionally contacting the Protein A chromatography column with a first wash solution,
(d) applying one or more wash solutions (such as 1, 2 or 3 wash solutions) to the Protein A chromatography column, wherein the wash solution comprises arginine or an arginine salt or other arginine derivative in a concentration of above 500 mM, and wherein the pH of the wash solution is greater than 8.0,
(e) optionally contacting the Protein A chromatography column in d) with another second wash solution,
(f) contacting the washed Protein A chromatography column in d) or e) with an elution solution under conditions suitable for eluting the product, and
(g) collecting an eluate comprising the product.

2. The method of claim 1, wherein the concentration of the arginine or the arginine salt or arginine derivative comprises:
(I) about 500 mM to about 600 mM;
(II) about 525 mM to about 575 mM,
(III) about 550 mM to about 575 mM or
(IV) is about 575 mM.

3. The method of claim 1, wherein the pH of the one or more wash solutions of (d):
(i) range from about 8.0 to about 8.7;
(ii) range from about 8.2 to about 8.7;
(iii) range about 8.5 to about 8.7;
(iv) is/are about 8.5;
(v) is/are about 8.6; or (vi) is/are about 8.7.

4. The method of claim 1, wherein the arginine salt or arginine derivative comprises Arg·HCl, acetyl arginine, agmatine, arginic acid, N-alpha-butyroyl-L-arginine, or N-alpha-pyvaloyl arginine.

5. The method of claim 1, wherein the monoclonal antibody is specific for CGRP, such as Eptinezumab.

6. The method of claim 1, wherein the monoclonal antibody is specific for PACAP, such as PACAP-1.

7. The method of claim 1, wherein the monoclonal antibody is specific for ACTH, such as ACTH-1.

8. The method of claim 1, wherein the concentration of arginine in the wash solution in d) is about 575 mM and PH is 8.5 or 8.7.

9. The method of claim 1, wherein the concentration of arginine in the one or more wash solutions in d) is about 525 mM and the pH is 8.5 or 8.7.

10. The method of claim 1, wherein one or more of the impurities are a host cell protein, a nucleic acid, a product variant, and/or an endotoxin.

11. The method of claim 1, wherein the elution solution comprises about 15 mM to about 50 mM Acetic Acid and optionally comprises about 20 mM to 50 mM glycine.

12. The method of claim 1, wherein the elution solution has a pH of between about 2 and about 5.

13. The method of claim 1, wherein the elution solution has a pH of about 4.

14. The method of claim 1, wherein the one or more wash solutions in c) and e) do not contain arginine or an arginine salt or arginine derivative.


SEQUENCE LISTING (32 sequences)

SEQ ID NO: 1 - protein, 5 aa, synthetic construct
GYYMN

SEQ ID NO: 2 - protein, 16 aa, synthetic construct
VIGINGATYYASWAKG

SEQ ID NO: 3 - skipped

SEQ ID NO: 4 - protein, 111 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 5 - protein, 440 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 441 aa, synthetic construct
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 13 aa, synthetic construct
QASQSVYHNTYLA

SEQ ID NO: 8 - protein, 7 aa, synthetic construct
DASTLAS

SEQ ID NO: 9 - protein, 13 aa, synthetic construct
LGSYDCTNGDCFV

SEQ ID NO: 10 - protein, 113 aa, synthetic construct
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 11 - protein, 219 aa, synthetic construct
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, synthetic construct
SYYMT

SEQ ID NO: 13 - protein, 16 aa, synthetic construct
FIDAGGDAYYASWAKG

SEQ ID NO: 14 - protein, 4 aa, synthetic construct
DLDL

SEQ ID NO: 15 - protein, 112 aa, synthetic construct
EVQLVESGGGLVQPGGSLRLSCAASGIDLNSYYMTWVRQAPGKGLEWIGFIDAGGDAYYASWAKGRFTISRDNSKNTVYLQMNSLRAEDTAVYFCARDLDLWGQGTLVTVSS

SEQ ID NO: 16 - protein, 441 aa, synthetic construct
EVQLVESGGGLVQPGGSLRLSCAASGIDLNSYYMTWVRQAPGKGLEWIGFIDAGGDAYYASWAKGRFTISRDNSKNTVYLQMNSLRAEDTAVYFCARDLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 12 aa, synthetic construct
QSSESVYGNYLA

SEQ ID NO: 18 - protein, 7 aa, synthetic construct
EASKLES

SEQ ID NO: 19 - protein, 10 aa, synthetic construct
AGGDISEGVA

SEQ ID NO: 20 - protein, 110 aa, synthetic construct
DIQLTQSPSTLSASVGDRVTITCQSSESVYGNYLAWFQQKPGKAPKFLIYEASKLESGVPSRFSGSGSGTEFTLTISSLQPDDFATYYCAGGDISEGVAFGGGTKVEIKR

SEQ ID NO: 21 - protein, 216 aa, synthetic construct
DIQLTQSPSTLSASVGDRVTITCQSSESVYGNYLAWFQQKPGKAPKFLIYEASKLESGVPSRFSGSGSGTEFTLTISSLQPDDFATYYCAGGDISEGVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 6 aa, synthetic construct
SGYDIC

SEQ ID NO: 23 - protein, 17 aa, synthetic construct
CIDTGSGNTYYASSAKG

SEQ ID NO: 24 - protein, 5 aa, synthetic construct
GISSI

SEQ ID NO: 25 - protein, 115 aa, synthetic construct
EVQLVESGGGLVQPGGSLRLSCAASGFTVSSGYDICWVRQAPGKGLEWIGCIDTGSGNTYYASSAKGRFTMSRDNSKNTVYLQMNSLRAEDTAVYYCAKGISSIWGQGTLVTVSS

SEQ ID NO: 26 - protein, 444 aa, synthetic construct
EVQLVESGGGLVQPGGSLRLSCAASGFTVSSGYDICWVRQAPGKGLEWIGCIDTGSGNTYYASSAKGRFTMSRDNSKNTVYLQMNSLRAEDTAVYYCAKGISSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 11 aa, synthetic construct
QASQTISSDLA

SEQ ID NO: 28 - protein, 7 aa, synthetic construct
AASKLTS

SEQ ID NO: 29 - protein, 12 aa, synthetic construct
QTYYDIIDDGAT

SEQ ID NO: 30 - protein, 111 aa, synthetic construct
DIQMTQSPSTLSASVGDRVTITCQASQTISSDLAWYQQKPGKAPKLLIYAASKLTSGVPSRFSGSGSGTEFTLTISSLQPDDFATYYCQTYYDIIDDGATFGGGTKVEIKR

SEQ ID NO: 31 - protein, 217 aa, synthetic construct
DIQMTQSPSTLSASVGDRVTITCQASQTISSDLAWYQQKPGKAPKLLIYAASKLTSGVPSRFSGSGSGTEFTLTISSLQPDDFATYYCQTYYDIIDDGATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 445 aa, synthetic construct
EVQLVESGGGLVQPGGSLRLSCAASGFTVSSGYDICWVRQAPGKGLEWIGCIDTGSGNTYYASSAKGRFTMSRDNSKNTVYLQMNSLRAEDTAVYYCAKGISSIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK